(19) United States
(12) Patent Application Publication
(10) Pub. No.: US 2005/0182136 A1
(43) Pub. Date: Aug. 18, 2005

(54) N-acetylcysteine compositions and methods for the treatment and prevention of endothelial dysfunction

(76) Inventor: Allen Jeremais, Newton, MA (US)

Correspondence Address: DOCKET ADMINISTRATOR;LOWENSTEIN SANDLER PC, 65 LIVINGSTON AVENUE, ROSELAND, NJ, 07068, US

(21) Appl. No.: 11/060,039

(22) Filed: Feb. 16, 2005

Related U.S. Application Data
(60) Provisional application No. 60/545,316, filed on Feb. 17, 2004.

(51) Int. Cl. A61K 31/198

(52) U.S. Cl. 514/562

(57) ABSTRACT

The invention provides compositions and methods for the treatment or prevention of endothelial dysfunction in mammals, including humans, which comprises an effective amount of N-acetylcysteine or a salt or derivative thereof either alone or in combination with a therapeutically effective amount of a therapeutic agent. The invention also provides compositions and methods for the treatment or prevention of endothelial dysfunction in mammals, including humans, which comprises an effective amount of N-acetylcysteine or a salt or derivative thereof either alone or in combination with a therapeutically effective amount of a therapeutic agent whose side effects are made worse by increased oxidative stress or treatment related decreases in a mammal's cysteine/glutathione levels or are otherwise relieved by administration of NAC.

### CROSS-REFERENCE TO RELATED APPLICATIONS

This application claims the benefit of U.S. Provisional Application No. 60/545,316, filed Feb. 17, 2004, the entire disclosure of which is hereby incorporated herein by reference.

### FIELD OF THE INVENTION

The present invention relates to compositions and methods for the treatment or prevention of endothelial dysfunction in mammals including humans. The compositions of this invention comprise an effective amount of N-acetylcysteine (NAC) or a pharmaceutically acceptable salt or derivative thereof alone or in combination with a therapeutically effective amount of a therapeutic agent, preferably in combination with a pharmaceutically acceptable carrier. The method of treating or preventing endothelial dysfunction in mammals including humans comprises administering an effective amount of NAC or a pharmaceutically acceptable salt or derivative thereof alone or in combination with a therapeutically effective amount of a therapeutic agent, preferably in combination with a pharmaceutically acceptable carrier.

### BACKGROUND OF THE INVENTION

The vascular endothelium plays a central role in regulating vascular smooth muscle and cardiac contractile function and structure via the release of vasoactive substances. These substances thus maintain a balance between dilation and constriction, growth inhibition and growth promotion, antithrombosis/antifibrinolysis and prothrombosis; antiinflammation and proinflammation, and antioxidant and pro-oxidant properties through the interaction of a variety of factors (1-3).

The endothelium maintains vascular tone by the production of opposing dilating and constricting factors. Prominent dilating factors in the physiologic state include nitric oxide (NO), bradykinin, and prostacyclin. (Luscher, T. F., Barton, M. Clin. Cardio. (1997): 10 (Suppl. II): 3-10; Vane, J. R., Anggard, E. E., Botting, R. M. New England J. Med. (1990) 323: 27-36). Growth inhibitors such as NO and bradykinin appear to outweigh those of growth promoters in healthy endothelium since the vessel wall is normally quiescent and does not exhibit smooth muscle cell proliferation (Luscher, T. F., Barton, M. Clin. Cardio. (1997): 10 (Suppl. II): 3-10). The fine balance that characterizes the normal fibrinolytic system is maintained primarily by plasminogen activator inhibitors and tissue-type plasminogen activators, both of which are produced by endothelial and smooth muscle cells (Vaughan, D. E. Clin. Cardiol. (1997) 20 (Suppl. II): 34-37). NO, bradykinin and prostacyclin have antithrombotic effects; bradykinin in particular has been shown to induce dose-dependent increases in plasma t-PA levels (Brown, N. J., Nadeau, J., Vaughan, D. E. Throm. Haemost. (1997) 77: 522-525).

Nitric oxide mediates smooth muscle relaxation by activating guanylate cyclase to increase intracellular concentrations of cyclic guanosine monophosphate (GMP), which in turn activates protein kinases that can regulate free calcium ion levels in the muscle cells and cause relaxation of smooth muscle by phosphorylating myosin light chain kinase. A short-lived free radical gas, NO is synthesized in the vascular endothelial cell from L-arginine by NO synthase. It diffuses to the smooth muscle cell where it activates the enzyme guanylate cyclase which leads to an increase in cyclic GMP and then muscle relaxation.

Normally, low levels of NO are continuously released by the vascular endothelium to keep blood vessels in a dilated state (Luscher, T. F., Barton, M. Clin. Cardio. (1997): 10 (Suppl. ID): 3-10). Endothelium-derived nitric oxide (NO) is released in response to acetylcholine, other receptor-dependent agonists, and physical factors such as shear stress. Besides inhibiting vascular smooth muscle cell growth, NO also has been implicated in preventing platelet adhesion to the endothelial surface, and endothelial cell/leukocyte interactions (Luscher, T. F., Barton, M. Clin. Cardio. (1997): 10 (Suppl. II): 3-10); Cooke, J. P., Tsao, P. S. Arterioscler Thromb. (1994) 14: 653-655). NO also may counterbalance the effects of oxygen-derived free radicals produced in cell metabolism (Harrison, D. G. Clin. Cardio. (1997) 20 (Suppl. II): 11-17).

The term endothelial dysfunction, which describes abnormalities in the action and metabolism of endothelium-derived nitric oxide which induce structural and functional abnormalities of the vascular endothelium, is involved in the pathophysiology of a number of conditions, such as atherosclerosis, hypertension, diabetes, left ventricular hypertrophy and heart failure. Endothelial cell damage, with loss of the vascular protective effects of NO, is viewed as a likely early step in the pathogenesis of the microvascular complications of diabetes, and has been described in association with risk factors for coronary artery disease (Vita, J. A. et al., J. Clin. Investig. (1998) 101(6): 1408-1414).

Although the molecular basis of endothelial cell dysfunction is not well understood, increased free radical generation, increased oxidative stress, and abnormal plasma lipid composition have all been implicated in macrovascular endothelial cell dysfunction. Increased oxidative stress appears to be associated with known risk factors for expression of coronary artery disease, such as hypertension, hypercholesterolemia, smoking and diabetes mellitus (Harrison, D. G. Clin. Cardio. (1997) 20 (Suppl. II): 11-17; Griendling, K. K., Alexander, R. W. Circulation (1997) 96: 3264-3265; Chin, J. H., Azhar, S., Hoffman, B. B. J. Clin. Investig. (1992) 89: 10-18; Galle, J. et al. Hypertension (1994) 23: 556-564;Freyschuss,. A., et al. Arterioscler. Thromb. Vasc. Biol. (1997) 17: 1178-1184; Rubenstein, I., et al. Am. J. Physiol. (1991) 261: H1913-1918). Reactive oxygen species such as superoxide anion are produced in response to these risk factors. Superoxide anion and other free radicals inactivate NO and induce cell growth, kinase activity and lipid peroxidation.

Glutathione (GSH), a tripeptide that is normally found in all animal cells and most plants and bacteria at relatively high (1-10 millimolar) concentrations, helps to protect cells against oxidative damage that would otherwise be caused by free radicals and reactive oxidative intermediates (ROIs) produced during cell metabolism or as the results of, for example, drug overdose. It is a major scavenger of reactive oxidative intermediates present in all eukaryotic forms of life and is generally required to protect cells against damage by oxidants. Glutathione reduces (and thereby detoxifies) intracellular oxidants and is consumed by this reaction. Glutathione is oxidized to the disulfide-linked dimer (GSSG), which is actively pumped out of cells and becomes largely unavailable for reconversion to reduced glutathione. Thus, unless glutathione is resynthesized through other pathways, utilization of this compound is associated with a reduction in the amount of glutathione available. The antioxidant effects of glutathione are also mediated less directly by the role of this compound in maintaining other antioxidants in reduced form. Thus, pharmaceutical compounds, such as N-acetylcysteine (NAC), that replenish or elevate glutathione levels work, at least in part, through enhancement of the defense mechanisms seemingly utilized to normally protect tissue from ROI mediated damage.

Published studies have suggested that intracellular GSH may modulate the production and/or bioactivity of NO although the mechanism(s) by which it may do so are not yet clear. GSH is known to prevent nitric oxide synthase inactivation which is thought to result from the formation of peroxynitrite due to simultaneous generation of NO and superoxide by the enzyme (Stuehr, D. J., N. S. Kwon, C. F. Nathan. Biochem. Biophys. Res. Commun. (1990) 168: 558-565; Hobbs, A. J., J. M. Kukoto, L. J. Ignarro. Proc. Nat. Acad. Sci. U.S.A. (1994) 91: 10992-10996). Glutathione also may modulate the action and metabolism of endothelial-derived NO by virtue of its central role in regulation of the intracellular redox state.

As previously described in published articles and issued patents, NAC is a source of sulhydryl groups which can stimulate glutathione synthesis, enhance glutathione-S-transferase activity, and/or promote detoxification by liver and lung tissue of some direct-acting mutagens. NAC has been used by many physicians for treatment of hepatic failure of any etiology, whether known or unknown, and is the accepted antidote for cyclophosphamide and acetaminophen poisoning.

The mechanisms through which NAC prevents or reverses toxicity are mainly thought to involve glutathione replenishment. However, additional mechanisms through which NAC works directly via the cysteine molecule itself are not excluded. Earlier studies have shown that NAC replenishes glutathione following acetaminophen overdose, which otherwise leads to a fatal depletion of glutathione in the liver. This non-toxic drug enters cells readily and replenishes the intracellular cysteine required to produce glutathione, thus leading to an increase in glutathione levels. It may be noted that the effectiveness of NAC depends on the presence of the reduced form, which may, for example, liberate the reduced form of glutathione from homo- and hetero-disulfide derivatives in thiol-disulfide exchange reactions.

In animal experiments, a daily NAC dose of 150 mg/kg was sufficient to substantially increase both glutathione and cysteine levels over no treatment (see Jeremias et al., (2001) Circulation, 104: 11-743). In HIV patients, in whom there is a high incidence of glutathione deficiency, subjects took 3,200 to 8,000 mg (mean 4,400 mg) of NAC daily to replenish glutathione levels (see Herzenberg et al., Proc. Nat'l Acad. Sci. 94: 1967-1972 (1997)). Several studies, which examined the use of oral NAC in reducing the renal toxicity of intravenous contrast agents, found that there was a significant reduction in the increase of serum creatinine (a measure of kidney function in which lower numbers of creatinine indicate better renal function) after the administration of the contrast agent, indicating a kidney protective function by NAC that is likely explained by decrease in oxidative stress (see Kay et al. (2003), JAMA 289: 553-558). In patients with end-stage renal failure who are prone to endothelial dysfunction, NAC treatment with 600 mg twice a day significantly reduced various adverse cardiovascular events (see Tepel et al., (2003) Circulation 107: 992-995).

Oral dietary supplements (particularly vitamins C and E), and probucol, a lipid lowering agent with antioxidant properties, among others, have been shown to have positive effects on vascular endothelial function (Freyschuss, A., et al. Arterioscler. Thromb. Vasc. Biol. (1997) 17: 1178-1184.; Klemsdal, T. O. et al. Cardiovasc. Res. (1994) 28: 1397-1402.; Zhang, J. et al. Microvasc Res (1999) 58: 305-311; Keaney, et al. J. Clin. Invest. (1995) 95: 2520-2529). Improved formulations and methods to treat or prevent endothelial dysfunction are of particular interest in view of the considerable economic cost of cardiovascular disease. The present invention addresses this problem.

### SUMMARY OF THE INVENTION

Compositions and methods of treatment are provided for administration of NAC or a salt or derivative thereof, alone or in combination with any therapeutic agent to treat or prevent endothelial dysfunction, including those drugs whose side effects are made worse by decreases in a mammal's (including human's) intracellular cysteine/gluathione levels or increased oxidative stress, or whose side effects are otherwise relieved by administration of NAC.

According to one embodiment of the invention, a composition useful for treating or preventing endothelial dysfunction in mammals including humans, comprising an effective amount of N-acetylcysteine or a pharmaceutically acceptable salt or derivative thereof alone or in combination with a therapeutically effective amount of a therapeutic agent, in combination with a pharmaceutically acceptable carrier is described. In another embodiment, the therapeutic agent of such compositions comprises at least one antihypertensive, or lipid lowering agent. In another embodiment, the therapeutic agent of such compositions comprises at least one antihypertensive, or lipid lowering agent that produces oxidative stress. In another embodiment, the therapeutic agent of such compositions comprises at least one antihypertensive, or lipid lowering agent that produces treatment-related decreases in a mammal's cysteine/glutathione levels. In another embodiment, the composition further comprises at least one dietary supplement.

In another embodiment, a method of treating or preventing endothelial dysfunction in mammals including humans in need thereof comprises administering to a mammal an effective amount of N-acetylcysteine or a pharmaceutically acceptable salt or derivative thereof, alone or in combination with a therapeutically effective amount of a therapeutic agent, in combination with a pharmaceutically acceptable carrier. In another embodiment, the method comprises parenterally administering at least about 1 mg N-acetylcysteine or a pharmaceutically acceptable salt or derivative thereof to the mammal alone or in combination with a therapeutically effective amount of a therapeutic agent, in combination with a pharmaceutically acceptable carrier. In another embodiment, the method comprises orally administering at least about 1 mg N-acetylcysteine or a pharmaceutically acceptable salt or derivative thereof to the mammal alone or in combination with a therapeutically effective amount of a therapeutic agent, in combination with a pharmaceutically acceptable carrier. In another embodiment, the method comprises rectally administering at least about 1 mg N-acetylcysteine or a pharmaceutically acceptable salt or derivative thereof to the mammal alone or in combination with a therapeutically effective amount of a therapeutic agent, in combination with a pharmaceutically acceptable carrier. In another embodiment, the method further comprises administering a therapeutically effective amount of at least one antihypertensive, or lipid lowering agent serially or in combination with the N-acetylcysteine or a pharmaceutically acceptable salt or derivative thereof. In another embodiment, the therapeutic agent of such method comprises at least one antihypertensive, or lipid lowering agent that produces oxidative stress. In another embodiment, the therapeutic agent of such method comprises at least one antihypertensive, or lipid lowering antiviral agent that produces treatment-related decreases in a mammal's cysteine/glutathione levels. In another embodiment, the method further comprising administering at least one dietary supplement.

### DETAILED DESCRIPTION OF THE INVENTION

The invention described herein provides for the administration of an effective amount of NAC or a pharmaceutically acceptable salt or derivative thereof, alone or in combination with a therapeutically active agent to relieve endothelial dysfunction in a mammal, including humans, including where the effects of such therapy in the mammal may be due to oxidative stress or treatment-related decreases in the mammal's cysteine/glutathione levels or are otherwise relieved by administration of NAC.

By a “pharmaceutically acceptable derivative” is meant any ester, or salt of such ester, of NAC or any other compound which, upon administration to the recipient, is capable of providing (directly or indirectly) NAC or an active metabolite or residue thereof.

The compositions and methods of treatment above described include providing the compositions in oral, parenteral, or suppository form for oral, parenteral, or rectal administration. It is preferred that the NAC or a pharmaceutically acceptable salt or derivative thereof be substantially free of sulfones or other chemicals that interfere with the metabolism of the co-administered drug in its bioactive form. It is also preferred that the NAC be substantially free of its oxidized form, di-N-acetylcysteine. It is preferred that the therapeutic agent, serially or co-administered, be in any form in which it is typically available and the composition should be prepared in a manner that substantially prevents oxidation of the NAC during preparation or storage. In one preferred aspect of the invention, a zinc salt can be added to the composition to slow down degradation of NAC. It may be noted that the effectiveness of NAC depends on the presence of the reduced form, which may, for example, liberate the reduced form of glutathione from homo- and hetero-disulfide derivatives in thiol-disulfide exchange reactions.

A typical unit dosage of NAC composition (alone or in combination with a therapeutic agent) may be a solution suitable for oral or intravenous administration; an effervescent tablet suitable for dissolving in water, fruit juice, or carbonated beverage and administered orally; a tablet taken from two to six times daily, or one time-release capsule or tablet taken once or twice a day and containing a proportionally higher content of active ingredient, etc. The time-release effect may be obtained by capsule materials that dissolve at different pH values, by capsules that release slowly by osmotic pressure, or by any other known means of controlled release. Unit dosage forms may be provided wherein each dosage unit, for example, teaspoonful, tablespoonful, gel capsule, tablet or suppository, contains a predetermined amount of the compositions of the present invention. Similarly, unit dosage forms for injection *or intravenous administration may comprise NAC in a composition as a solution in sterile water, normal saline or another acceptable carrier. The specifications for the unit dosage forms of the present invention depend on the effect to be achieved and the intended recipient.

It is preferred that the compositions according to the present invention contain from about 1 mg of NAC per dosage unit, preferably at least 3 mg to 2,000 mg per dosage unit for oral administration, and 20-20,000 mg for parenteral. Suppositories are formulated in the manner well known in the art and usually comprise at least about 1 mg NAC per dosage unit and may be as high as about 20,000 mg, more usually not more than about 2,000 mg together with effective amounts of a therapeutic agent. Oral liquid dosage forms usually comprise at least about 1 mgl NAC, and may be as high as about 20,000 mg, usually not more than about 2,000 mg, together with effective amounts of a therapeutic agent.

The unit dose of NAC, in combination with a therapeutically effective agent, or alone for the treatment of symptoms of endothelial dysfunction, will usually comprise at least about 1 mg/kg to a maximum amount of 70 mg/kg, usually at least about 200 mg (for adult doses); and usually not more than about 3,600 mg administered two or three times daily. This amount may be varied at the physician's discretion. Patients on therapy known to deplete cysteine/glutathione or produce oxidative stress may benefit from higher amounts of NAC.

Over-the-counter NAC can be variably produced and packaged. Because the production and packaging methods generally do not guard against oxidation, the NAC can be significantly contaminated with bioactive oxidation products. These may be particularly important in view of data indicating that the oxidized form of NAC has effects counter to those reported for NAC and is bioactive at doses roughly 10-100 fold less than NAC (see Samstrand et al J. Pharmacol. Exp. Ther. 288: 1174-84 (1999)).

The distribution of the oxidation states of NAC as a thiol and disulfide depends on the oxidation/reduction potential. The half-cell potential obtained for the NAC thiol/disulfide pair is about +63 mV, indicative of its strong reducing activity among natural compounds (see Noszal et al. J. Med. Chem. 43:2176-2182 (2000)). In a preferred embodiment of the invention, the preparation and storage of the formulation is performed in such a way that the reduced form of NAC is the primary form administered to the patient. Storage in cool dark environments is also preferred.

The determination of reduced and oxidized species present in a sample may be determined by various methods known in the art, for example with capillary electrophoresis, HPLC, etc. as described by Chassaing et al. J Chromatogr B Biomed Sci Appl 735(2):219-27 (1999).

The compositions of the present invention may be administered orally, parenterally, or rectally in dosage unit formulations containing conventional nontoxic pharmaceutically acceptable carriers, adjuvants, and vehicles as desired. The term parenteral as used herein includes subcutaneous injections, intravenous, intramuscular, intrasternal injection, or infusion techniques. Topical administration may also involve the use of transdermal administration such as transdermal patches or iontophoresis devices that are prepared according to techniques and procedures well known in the art.

Solid dosage forms for oral administration may include capsules, tablets, pills, powders, granules and gels. In such solid dosage forms, the active compounds may be admixed with at least one inert diluent such as sucrose, lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Suppositories for rectal administration of the drug composition, such as for treating pediatric fever etc., can be prepared by mixing the drug with a suitable nonirritating excipient such as cocoa butter and polyethylene glycols which are solid at ordinary temperatures but liquid at the rectal temperature and will therefore melt in the rectum and release the drug.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium.

The compositions of this invention can further include conventional carriers and excipients, i.e., pharmaceutically acceptable organic or inorganic carrier substances suitable for the particular route of administration which do not deleteriously react with the active compounds. Suitable pharmaceutically acceptable carriers include, but are not limited to, water, salt solutions, alcohol, vegetable oils, polyethylene glycols, gelatin, lactose, amylose, magnesium stearate, talc, silicic acid, viscous paraffin, perfume oil; fatty acid monoglycerides and diglycerides, petroethral fatty acid esters, hydroxymethylcellulose, polyvinylpyrrolidone, etc. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances that increase the viscosity of the suspension and include, for example, sodium carboxymethyl cellulose, sorbitol and/or dextran. Optionally, the suspension may also contain stabilizers.

The composition, if desired, can also contain minor amounts of wetting or emulsifying agents or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc.

The therapeutically active agent of the present invention can be formulated per se or in salt form. Pharmaceutically acceptable salts include, but are not limited to, those formed with free amino groups such as those derived from hydrochloric, phosphoric, sulfuric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of compounds in the compositions of the present invention which will be effective in the treatment of a particular disorder or condition will depend on the nature of the disorder or condition, and can be determined by standard clinical techniques. See, for example, Goodman and Gilman; The Physician's Desk Reference, Medical Economics Company, Inc., Oradell, N.J. (1995); and Drug Facts and Comparisons, Facts and Comparisons, Inc., St. Louis, Mo. (1993). The precise dose to be employed in the formulation will also depend on the route of administration, and the seriousness of the disease or disorder, and should be decided according to the judgment of the practitioner and each patient's circumstances.

The present invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirit and scope of the present invention. All such modifications are intended to be within the scope of the claims appended hereto.

1. A composition useful for treating or preventing endothelial dysfunction in mammals, including humans, comprising an effective amount of N-acetylcysteine, or a pharmaceutically acceptable salt of N-acetylcysteine or a derivative of N-acetylcysteine alone or in combination with a therapeutically effective amount of a therapeutic agent, in combination with a pharmaceutically acceptable carrier.

2. The composition according to claim 1, wherein a dosage unit of the composition contains at least about 1 mg N-acetylcysteine or a pharmaceutically acceptable salt of N-acetylcysteine or a derivative of N-acetylcysteine.

3. The composition according to claim 1, wherein the therapeutic agent comprises at least one antihypertensive or lipid-lowering agent.

4. The composition according to claim 1, wherein the therapeutic agent is at least one antihypertensive or lipid lowering agent that produces oxidative stress.

5. The composition according to claim 1, wherein the therapeutic agent is at least one antihypertensive or lipid lowering agent that produces treatment-related decreases in a mammal's cysteine/glutathione levels.

6. The composition according to claim 1, further comprising at least one dietary supplement.

7. A method of treating or preventing endothelial dysfunction in mammals, including humans, comprising the step of administering to a mammal an effective amount of N-acetylcysteine or a pharmaceutically acceptable salt of N-acetylcysteine or a derivative of N-acetylcysteine alone or in combination with a therapeutically effective amount of a therapeutic agent, in combination with a pharmaceutically acceptable carrier.

8. The method according to claim 8, further comprising the step of parenterally administering at least about 1 mg N-acetylcysteine or a pharmaceutically acceptable salt of N-acetylcysteine or a derivative of N-acetylcysteine to the mammal.

9. The method according to claim 8, further comprising the step of orally administering at least about 1 mg N-acetylcysteine or a pharmaceutically acceptable salt of N-acetylcysteine or a derivative of N-acetylcysteine to the mammal.

10. The method according to claim 8, further comprising the step of rectally administering at least about 1 mg N-acetylcysteine or a pharmaceutically acceptable salt of N-acetylcysteine or a derivative of N-acetylcysteine to the mammal.

11. The method according to claim 8, further comprising the step of administering a therapeutically effective amount of at least one antihypertensive or lipid-lowering agent serially or in combination with the N-acetylcysteine or the pharmaceutically acceptable salt of N-acetylcysteine or a derivative of N-acetylcysteine.

12. The method according to claim 12, wherein the antihypertensive or lipid-lowering agent produces oxidative stress.

13. The method according to claim 12, wherein the antihypertensive or lipid-lowering agent produces treatment-related decreases in the mammal's cysteine/glutathione levels.

14. The method according to claim 8, further comprising administering at least one dietary supplement.
